(19) United States
(12) Patent Application Publication
(10) Pub. No.: US 2013/0231592 A1
(43) Pub. Date: Sep. 5, 2013

(54) SKIN CARE APPARATUS

(71) Applicant: BOMTECH ELECTRONICS CO., LTD., Seoul (KR)

(72) Inventor: Jong-dae Lee, Seoul (KR)

(73) Assignee: BOMTECH ELECTRONICS CO., LTD., Seoul (KR)

(21) Appl. No.: 13/646,230

(22) Filed: Oct. 5, 2012

(30) Foreign Application Priority Data
Mar. 5, 2012 (KR) 10-2012-0022169

(51) Int. Cl. A61N 7/00

(52) U.S. Cl. 601/2

(57) ABSTRACT

A skin care apparatus using ultrasonic vibration is provided. The skin care apparatus includes a main body, an ultrasonic generation portion installed in the main body to generate vibration through ultrasonic waves, and a vibration member transferring ultrasonic vibration generated from the ultrasonic generation portion to a skin and having a space portion through which body wastes existing in the skin are discharged out of the skin by the ultrasonic vibration.

### CROSS REFERENCE TO RELATED APPLICATIONS

This application claims priority under 35 U.S.C. §119(a) to Korean Patent Application No. 10-2012-0022169, filed on Mar. 5, 2012, in the Korean Intellectual Property Office, the disclosure of which is incorporated in its entirety herein by reference.

### BACKGROUND OF THE INVENTION

1. Field of the Invention

The present invention relates to a skin care apparatus, and more particularly to a skin care apparatus which can discharge body wastes that are included in a skin to outside and improve the blood circulation by stimulating the skin through ultrasonic vibration.

2. Description of the Related Art

Recently, a skin massage tool which can massage and clean a facial skin using ultrasonic vibration and cure fatness of an abdominal region has been developed and used.

The skin massage tool is helpful to discharge the body wastes included in the skin out of the skin and to perform the smooth circulation of the blood by stimulating the skin through transfer of the ultrasonic vibration to the skin. The skin massage tool is typically provided with an ultrasonic generation member that can generate the ultrasonic vibration, and a vibration transfer member transferring the ultrasonic vibration from the ultrasonic generation member to the skin. For example, skin massage tools using ultrasonic vibration in the related art are disclosed in Korean Registered Utility Model No. 265691 and Korean Registered Patent No. 830530.

In the case of the skin massage tool disclosed in Korean Registered Utility Model No. 265691, a contact portion that is in contact with the skin is in the form of a circular plate. Accordingly, since body wastes discharged from a skin portion being massaged are pressed by the circular contact portion, it is difficult to smoothly discharge the body wastes, and thus the massage effect may be considerably deteriorated.

Further, in Korean Registered Patent No. 830530, a vibration member that is in contact with the skin is in the form of a plate with a predetermined thickness. Accordingly, in the case of using the skin massage tool, one side of the vibration member should be kept in contact with the skin. Due to such a utilization method, the massage tool has the problem that it is required to frequently change the angle at which the vibration member makes contact with the skin portion being massaged depending on various bends of the skin portion being massaged to cause inconvenience in use.

### SUMMARY OF THE INVENTION

The present invention has been made to address at least the above problems and/or disadvantages and to provide at least the advantages described below. Accordingly, an aspect of the present invention provides a skin care apparatus which can smoothly discharge body wastes that are included in a skin out of the skin by ultrasonic vibration through providing of a plurality of skin contact projections of a vibration member that are arranged at predetermined intervals.

Another aspect of the present invention provides a skin care apparatus which can save the trouble of adjusting a vibration member at specified angles with respect to various bends of a skin portion which differ depending on the skin portion being massaged through a radially repeated arrangement or dispersed arrangement of a plurality of skin contact projections.

According to one aspect of the present invention, a skin care apparatus includes a main body; an ultrasonic generation portion installed in the main body to generate vibration through ultrasonic waves; and a vibration member transferring ultrasonic vibration generated from the ultrasonic generation portion to a skin and having a space portion through which body wastes existing in the skin are discharged out of the skin by the ultrasonic vibration.

The vibration member may be provided with a tube-shaped skin contact projection that is in contact with the skin.

It is also possible that the vibration member is provided with a plurality of tube-shaped skin contact projections that are in contact with the skin. In this case, the plurality of tube-shaped skin contact projections may be radially and repeatedly arranged at predetermined intervals from a center of the vibration member. Further, it is also possible that the plurality of tube-shaped skin contact projections are dispersedly arranged at predetermined intervals.

The tube-shaped skin contact projection may be in the form of any one of a cylinder, an ellipse, and a polygon.

### BRIEF DESCRIPTION OF THE DRAWINGS

The above and other aspects, features and advantages of the present invention will be more apparent from the following detailed description when taken in conjunction with the accompanying drawings, in which:

FIGS. 1 and 2 are perspective views illustrating a skin care apparatus according to a first embodiment of the present invention;

FIG. 3 is a partially cut away side view illustrating a skin care apparatus according to the first embodiment of the present invention; and

FIG. 4 is a perspective view illustrating a skin care apparatus according to a second embodiment of the present invention.

### DETAILED DESCRIPTION OF EMBODIMENTS OF THE PRESENT INVENTION

Hereinafter, a skin care apparatus according to first and second embodiments of the present invention will be described in detail with reference to the accompanying drawings. For reference, in explaining the present invention, detailed descriptions of constructions or processes known in the art may be omitted to avoid obscuring the subject matter of the present invention.

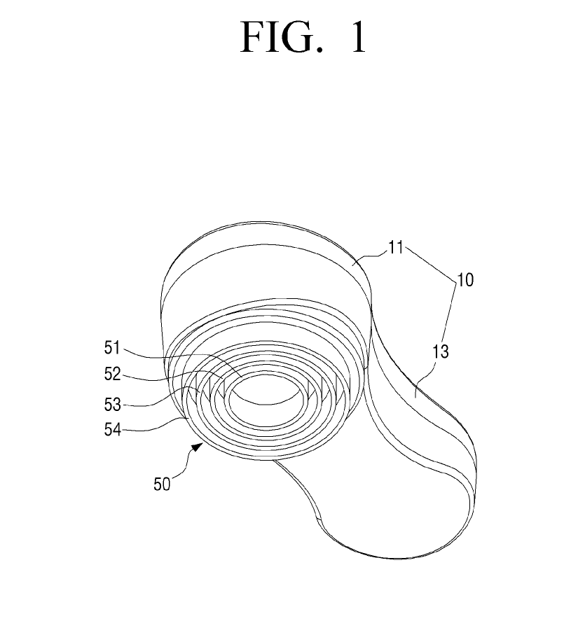

Referring to FIGS. 1 to 3, a skin care apparatus according to a first embodiment of the present invention includes a main body 10, an ultrasonic generation portion 30, and a vibration member 50.

The main body 10 includes a head portion 11 on which the ultrasonic generation portion 30 and the vibration member 50 are installed, and a gripping portion 13 formed to extend from one side of the head portion 11.

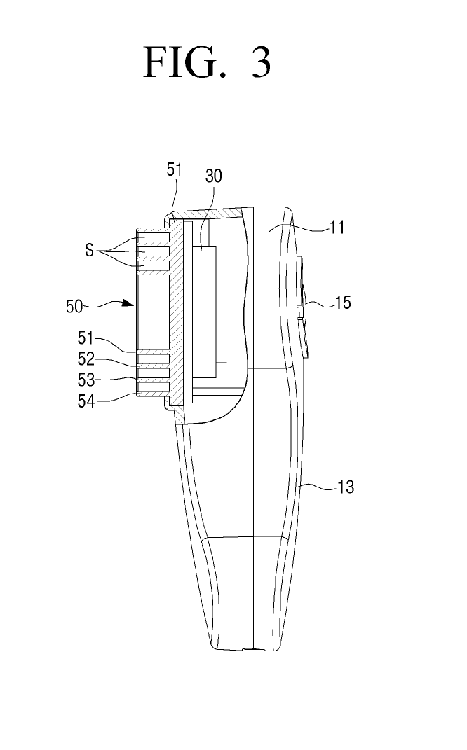

On the inside of the head portion 11, as shown in FIG. 3, the ultrasonic generation portion 30 is installed, and the vibration member 50 is installed to be in close contact with the ultrasonic generation portion 30. In this case, the vibration member 50 directly receives ultrasonic vibration generated from the ultrasonic generation portion 30 and transfers the ultrasonic vibration to a skin. Further, an operation button 15 is installed on an opposite side of the head portion 11 to the side where the vibration member 50 is installed. The operation button 15 corresponds to a power button for turning on/off the ultrasonic generation portion 30.

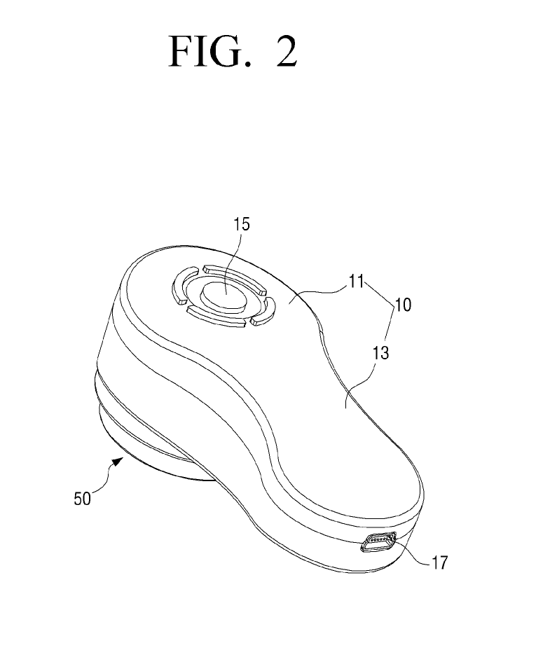

The gripping portion 13 is a portion that a user can grip, and a PCB (not illustrated) on which a plurality of electronic elements are mounted is installed inside the gripping portion 13. Further, as shown in FIG. 2, a power supply terminal 17 for supplying an external power supply to the inner PCB is installed on the gripping portion 13. In this case, the terminal may be a USB terminal, and in this case, the power can be easily supplied from a neighboring computer.

The ultrasonic generation portion 30 receives the power supply from the PCB and generates ultrasonic vibration. One side of the ultrasonic generation portion 30 is in surface contact with one side surface of the vibration member 50 to transfer the ultrasonic vibration to the vibration member 50.

A part of the vibration member 50 is coupled to the head portion 11, and thus the vibration member 50 does not secede from the head portion 11 even during the generation of the ultrasonic vibration. On the vibration member 50, a plurality of cylinder-shaped skin contact projections 51 to 54 having predetermined lengths toward skin contact portions thereof are repeatedly formed.

That is, the plurality of cylinder-shaped skin contact projections 51 to 54, which have different diameters that become gradually larger toward an outside of the vibration member 50, are arranged in a manner that the skin contact projections 52 to 54 are arranged at predetermined intervals to surround the skin contact projection 51 that is formed to project from the center of the vibration member 50 in order.

In this case, a space portion S is formed between the plurality of skin contact projections 51 to 54, and serves not to disturb the discharge of the body wastes so that the body wastes included in the skin can be naturally discharged out of the skin by the ultrasonic vibration when the vibration member 50 massages the skin.

Further, since the plurality of cylinder-shaped skin contact projections 51 to 54 are radially arranged, a user can easily perform the skin massage in a state where the user naturally grips the skin care apparatus without the trouble of adjusting the vibration member 50 at specified angles with respect to various bends of the skin portion which differ depending on the skin portion being massaged.

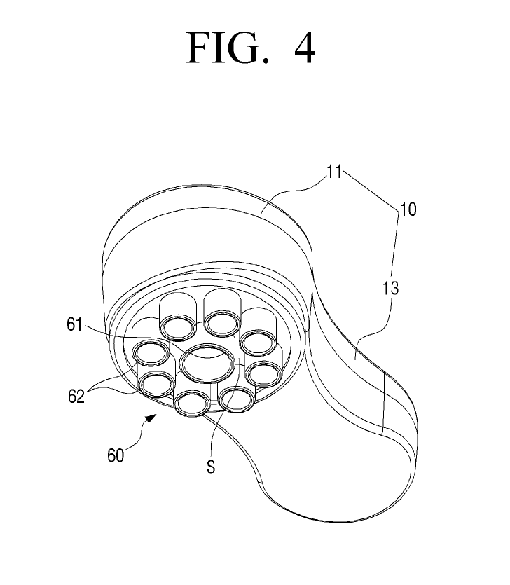

Referring to FIG. 4, the remaining configuration of the skin care apparatus except for a vibration member 60 according to a second embodiment of the present invention is mostly the same as the skin care apparatus according to the first embodiment. Accordingly, the description of the duplicate configuration according to the first embodiment will be omitted, and only the vibration member 60 will be described.

In the vibration member 60, as shown in FIG. 4, any one of a plurality of skin contact projections 61 and 62 is arranged in the center of the vibration member 60, and other skin contact projections 62 are dispersedly arranged around the skin contact projection 61. Even in this case, since the plurality of skin contact projections 61 and 62 are arranged to be spaced apart from each other so that a predetermined space portion S is formed between the skin contact projections 61 and 62, the body wastes included in the skin can be naturally discharged out of the skin in the space portion S by the ultrasonic vibration.

On the other hand, in the above-described first and second embodiments, it is exemplified that the plurality of skin contact projections 51 to 54 of the vibration member 50 are in the form of a cylinder. However, the shape of the skin contact projections is not limited thereto, but may be in various forms such as an ellipse or a polygon.

While the invention has been shown and described with reference to certain embodiments thereof, it will be understood by those skilled in the art that various changes in form and detail may be made therein without departing from the spirit and scope of the present invention, as defined by the appended claims.

What is claimed is:

1. A skin care apparatus comprising:
a main body;
an ultrasonic generation portion installed in the main body to generate vibration through ultrasonic waves; and
a vibration member installed on one side of the main body to transfer ultrasonic vibration generated from the ultrasonic generation portion to a skin,
wherein the vibration member is in the form of a tube to transfer the ultrasonic vibration to the skin in a state where a contact portion with the skin is minimized and a non-contact portion with the skin is maximized.

2. The skin care apparatus as claimed in claim 1, wherein the vibration member is provided with a plurality of tube-shaped skin contact projections that are in contact with the skin.

3. The skin care apparatus as claimed in claim 2, wherein the plurality of tube-shaped skin contact projections are radially and repeatedly arranged at predetermined intervals from a center of the vibration member.

4. The skin care apparatus as claimed in claim 2, wherein the plurality of tube-shaped skin contact projections are dispersedly arranged at predetermined intervals.

5. The skin care apparatus as claimed in claim 2, wherein the tube-shaped skin contact projection is in the form of any one of a cylinder, an ellipse, and a polygon.
